(19) United States
(12) Patent Application Publication
(10) Pub. No.: US 2016/0136130 A1
(43) Pub. Date: May 19, 2016

(54) Oral Ultraviolet Resistance Enhancer

(71) Applicant: KAO CORPORATION, Tokyo (JP)

(72) Inventors: Tetsuya KUWANO, Utsunomiya-shi, Tochigi (JP); Daiji KAGAWA, Utsunomiya-shi, Tochigi (JP); Takatoshi MURASE, Haga-gun, Tochigi (JP)

(73) Assignee: Kao Corporation, Chuo-ku, Tokyo (JP)

(21) Appl. No.: 14/899,415

(22) PCT Filed: Jul. 2, 2014
(86) PCT No.: PCT/JP2014/067674

§ 371 Date: Dec. 17, 2015

(30) Foreign Application Priority Data
Jul. 5, 2013 (JP) 2013-141982

(51) Int. Cl. A61K 31/366

(52) U.S. Cl. CPC A61K 31/366

(57) ABSTRACT

To provide an ultraviolet resistance enhancer capable of enhancing resistance of the skin to ultraviolet rays by oral ingestion, thereby reducing or suppressing skin damages caused by exposure to ultraviolet rays. An oral ultraviolet resistance enhancer comprising glucono-δ-lactone as an active ingredient.

### FIELD OF THE INVENTION

The present invention relates to an oral ultraviolet resistance enhancer capable of enhancing an ultraviolet resistance of skin by oral ingestion thereof.

### BACKGROUND OF THE INVENTION

Skin disorders, caused by increased ultraviolet rays resulting from a decreased ozone layer as one factor, have recently come into problem. For example, exposure to ultraviolet rays causes generation of erythema or edemas on the skin, formation of pigmentation, exacerbation of chloasma or ephelides, a decreased stratum corneummoisture content, reduced skin barrier function, reduced skin elasticity and formation of wrinkles accompanied therewith, photoaging such as a solar elastosis or cutis rhomboidalisnuchae, and further skin tumors.

For treating these skin disorders causedbyultraviolet rays, it has been attempted to enhance a resistance of the skin to ultraviolet rays by oral ingestion of a material, not a treatment or prevention with an external preparation as conventionally performed. It is reported, for example, that when bacteria of the genus Lactobacillus is ingested, the damage of the skin barrier function, caused by the irradiation of ultraviolet rays is suppressed (Patent Literature 1). It is also reported that when a composition in which elastin or ceramide is admixed with carotenoid is ingested, the erythema on the skin, induced by ultraviolet rays, can be effectively suppressed (Patent Literature 2).

Glucono-δ-lactone (GDL), which is a gluconic acid anhydride, is a sugar lactone in which a hydroxyl group at the 1-position of glucose is substituted by a ketone. Glucono-δ-lactone is converted from a glucose by an action of glucose-1-dehydrogenase in vivo, and the 6-phosphoric acid derivative thereof is a metabolic intermediate in the pentose phosphate cycle. Glucono-δ-lactone and gluconic acid are both assigned as a medicine or food additive in our country, and they are used, for example, as a stabilizer, a flavor, a pH-adjuster, an adhesive, an acidulant, a swelling agent, and a coagulant of tofu.

Recently, it has been reported that when gluconic acid is applied to the skin, IGF-1 secretion is promoted to exhibit effects of promoting hair-growth, reducing wrinkles on the skin and sagging skin, and the like (Patent Literature 3). It is also reported that when glucono-δ-lactone is applied to the skin of mouse, the skin surface becomes acidic to strengthen a stratum corneum structure, thereby improving a barrier function (Non Patent Literature 1).

It has not hitherto been known, however, what effects are generated on the skin disorders caused by ultraviolet rays, when glucono-δ-lactone or gluconic acid is orally ingested.

Patent Literature 1: JP 2008-179601 A

Patent Literature 2: JP 2004-229611 A

Patent Literature 3: JP 2008-100943 A

Non Patent Literature 1: Journal of investigative dermatology 2010; 130: 500-510

### SUMMARY OF INVENTION

The present invention relates to the following 1) to 4).
1) An oral ultraviolet resistance enhancer comprising glucono-δ-lactone as an active ingredient.
2) A non-therapeutic method for enhancing an ultraviolet resistance comprising: orally administering or ingesting an effective amount of glucono-δ-lactone.
3) Use of glucono-δ-lactone for producing an oral ultraviolet resistance enhancer.
4) A method for enhancing an ultraviolet resistance comprising: orally administering or ingesting an effective amount of glucono-δ-lactone.

### BRIEF DESCRIPTION OF DRAWINGS

FIG. 1 illustrates graphs showing an effect of suppressing ultraviolet-induced erythema and pigmentation by glucono-δ-lactone.

FIG. 2 illustrates graphs showing an anti-inflammatory (suppression of inflammatory cytokine) effect by glucono-δ-lactone.

FIG. 3 illustrates graphs showing an anti-inflammatory (suppression of an adhesion molecule involving lymphocytic infiltration) effect by glucono-δ-lactone.

FIG. 4 illustrates graphs showing an effect of suppressing melanin synthesis-related molecule by glucono-δ-lactone.

FIG. 5 illustrates graphs showing an effect of suppressing proliferation-related molecule by glucono-δ-lactone.

FIG. 6 illustrates graphs showing an effect of suppressing dermal denaturation-related molecule by glucono-δ-lactone.

FIG. 7 illustrates graphs showing an effect of suppressing pigmentation by glucono-δ-lactone.

FIG. 8 illustrates graphs showing an effect of suppressing reduction of skin barrier function by glucono-δ-lactone.

FIG. 9 illustrates graphs showing an effect of suppressing decrease of stratum corneum moisture content by glucono-δ-lactone.

FIG. 10 illustrates graphs showing an effect of suppressing reduction of a skin elasticity by glucono-δ-lactone.

FIG. 11 illustrates graphs showing effects of enhancing an ultraviolet resistance and suppressing UVB-induced erythema and pigmentation by glucono-δ-lactone in human tests.

FIG. 12 illustrates graphs showing effect of enhancing an ultraviolet resistance and suppressing ultraviolet ray-induced erythema and pigmentation by a combined formulation including glucono-δ-lactone and vitamins in human tests.

### DETAILED DESCRIPTION OF THE INVENTION

The present invention relates to provision of an oral ultraviolet resistance enhancer capable of increasing a resistance of the skin to ultraviolet rays by an oral ingestion, thereby reducing or suppressing skin damages caused by exposure to ultraviolet rays. The invention also relates to provision of a non-therapeutic method of enhancing an ultraviolet resistance comprising: orally administering or ingesting the ultraviolet resistance enhancer.

The present inventors investigated orally ingestible materials to enhance the ultraviolet resistance of the skin. As a result, they found that when glucono-δ-lactone is orally ingested, onset of erythema on the skin and the increase of epidermal thickness, caused by exposure to ultraviolet rays, were suppressed. They further found that expressions of inflammation-related molecules, melanin synthesis-related molecules, proliferation-related molecules, and dermal degeneration-related factors, which are exhibited and induced by the ultraviolet rays, were suppressed, and thus that glucono-δ-lactone is useful as an oral ultraviolet resistance enhancer.

The ultraviolet resistance enhancer of the present invention is useful, by ingestion, for reducing or suppressing photoaging and various skin disorders including, for example, skin inflammation such as erythema and edemas, formation of pigmentation, exacerbation of chloasma, ephelides and the like, reduction of stratum corneum function, a reduction of a skin barrier function, reduction of a skin elasticity and formation of wrinkles accompanied therewith, solar elastosis, cutis rhomboidalis nuchae, skin tumors and the like, which are caused by exposure of the skin to ultraviolet rays.

Glucono-δ-lactone (glucono-1,5-lactone), used in the ultraviolet resistance enhancer of the present invention, is an intramolecular ester obtained by removing one molecule of water removed from gluconic acid. When glucono-δ-lactone is dissolved in water, it changes gradually to gluconic acid, and the solution reaches an equilibrium state of glucono-δ-lactone and gluconic acid. In the present invention, accordingly, it is preferable to use glucono-δ-lactone, but it is possible to use gluconic acid. When gluconic acid is used, it is possible to use nontoxic salts thereof. Such salts may include, for example, salts with an alkali metal such as sodium or potassium, and salts with an alkaline earth metal such as calcium or magnesium.

Glucono-δ-lactone or gluconic acid may be produced by a known method, for example, a reaction of glucose in the presence of an organic solvent together with molecular oxygen using a palladium catalyst (JP 55-40606 A). It is also possible to produce gluconic acid liquid by an oxidative fermentation of glucose using some kind of mold (for example, Penicillium luteum purpurogenum, Penicillium chrysogenum, or Aspergillus niger) or bacterium (for example, Bacterium suboxydans, or Bacterium puridum), and gluconic acid liquid is concentrated under a reducedpressure, whereby glucono-δ-lactone can be produced (The eighth edition, Handbook of Japanese Standards of Food Additives (Hirokawa-Shoten Ltd.)). It is also possible to purchase and use a commercial product of glucono-δ-lactone or gluconic acid, which is commercially available as a pharmaceutical additive or food additive.

As shown in Examples described below, when a human orally ingests glucono-δ-lactone and then ultraviolet rays are irradiated to the skin, the onset of erythema and the pigmentation on the skin, caused by the irradiation of ultraviolet rays, can be suppressed. In addition, when glucono-δ-lactone is orally ingested to a mouse and then ultraviolet rays are irradiated to the skin, expressions of inflammation-related molecules (IL-1β, IL-6, GM-CSF, TNFα, COX-2, TLR3, SOCS3, VCAM-1, ICAM-1 and E-selectin), melanin synthesis-related molecules (EDN1, c-Kit, LIF, FGF2 andHGF), proliferation-relatedmolecules (PCNA and Cyclin D), dermal degeneration-related factors (collagenase (MMP 13), gelatinase (MMP2 and MMP9), and membrane-type MMP (MMP 14)), which are expressed and induced by the ultraviolet rays, are suppressed.

When ultraviolet rays are irradiated to the skin, expression of an inflammatory cytokine such as IL-1β, IL-6 or TNFα is increased, and expression of COX-2 is induced. As a result, prostaglandin E2 (PGE 2) is generated, vasodilation occurs and blood flow is increased, thus resulting in appearance of redness on the skin. Endothelin (EDN 1), SCF, LIF, FGF 2, HGF, GM-CSF and the like are generated from keratinocyte by the irradiation of ultraviolet rays, in addition to the inflammatory cytokine, and they are bonded to receptors on a cell membrane of melanocyte (for example, c-Kit: SCF receptor), to promote a melamine synthesis (Pigment Cell Research. 2004; 18: 2-12, The FASEB Journal. 2007; 21: 976-994). As a result of thus promotion of the melamine synthesis by irradiation of ultraviolet rays, the pigmentation is caused on the skin. In addition, a DNA synthesis such as PCNA or Cyclin D1 and expression of proliferation-related molecules, which involves the cell cycle, are also induced by irradiation of ultraviolet rays, and cell proliferation is activated and epidermal thickness is increased. Then, reduction of skin barrier function and decrease of stratum corneum moisture content are caused. Meanwhile, MMP (matrix metalloproteinase), induced by the irradiation of ultraviolet rays, decomposes a corium matrix such as collagen or elastin. For that reason, reduction of skin viscoelasticity and a formation of wrinkles accompanied therewith, what is called photoaging, is causedby chronic irradiation of ultraviolet rays. Furthermore, DNA damage is induced by the ultraviolet rays; when the repair thereof is not normally performed and the cell proliferation occurs, then the skin tumor is generated.

Accordingly, the suppressions of expressions of the inflammation-related molecules, the melanin synthesis-related molecules, the proliferation-related molecules and the dermal degeneration-related molecules due to an oral ingestion of glucono-δ-lactone show that glucono-δ-lactone is useful for reducing or suppressing the skin disorders such as skin aging or skin deterioration induced by the exposure to ultraviolet rays including, for example, skin inflammation such as erythema or edemas on the skin, pigmentation, reduction of skin barrier function, reduction of stratum corneum function, reduction of skin elasticity and formation of wrinkles accompanied therewith.

As described above, glucono-δ-lactone can be used for increasing the resistance of the skin to ultraviolet rays, i.e., can be used as an ultraviolet resistance enhancer. In addition, glucono-δ-lactone can be used for producing the ultraviolet resistance enhancer. Use of the ultraviolet resistance enhancer can be that for humans or non-human animals (orally administration or ingestion), and may be therapeutical or non-therapeutical. Here, the term “non-therapeutic” is a concept including no medical practices, i.e., a concept including no operating, treating or diagnosing method of a human, more specifically a concept including no operating, treating or diagnosing method of a human by a doctor or a person who receives directions from a doctor.

Accordingly, a composition comprising the oral ultraviolet resistance enhancer of the present invention is an oral pharmaceutical product, a quasi-drug, a supplement or a food product for enhancing the resistance of the skin to ultraviolet rays, i.e., the oral ultraviolet resistance enhancer is useful as a material or a drug formulation for adding to the oral pharmaceutical product, the quasi-drug, the supplement or the food product. The composition comprising the oral ultraviolet resistance enhancer of the present invention is an oral pharmaceutical product, a quasi-drug, a supplement or a food product for reducing or suppressing photoaging or skin disorder induced by exposure to ultraviolet rays including, for example, skin inflammation such as erythema or edemas on the skin, formation of pigmentation, exacerbation of chloasma or ephelides, reduction of stratum corneum function, reduction of skinbarrier function, reduction of skin elasticity and formation of wrinkles accompanied therewith, solar elastosis, cutis rhomboidalis nuchae, and skin tumor, i.e., the oral ultraviolet resistance enhancer is useful as a material or a drug formulation for adding to the oral pharmaceutical product, the quasi-drug, the supplement or the food product.

The pharmaceutical product, the quasi-drug or the supplement may have any dosage form of a solid formulation and a liquid formulation, and examples thereof may include a tablet, a coated tablet, a capsule, a granule, a pulvis, a powder, a sustained-release formulation, a suspension, an emulsion, internal liquid, sugar-coated tablet, a pill, a fine granule, syrup, elixir, etc.

The drug formulation described above may include pharmaceutically acceptable carriers. The carriers may include, for example, an excipient, a binder, a disintegrator, a lubricant, a diluent, an osmotic pressure regulator, a flow promotor, an absorption adjuvant, a pH-adjuster, an emulsifier, a preservative, a stabilizer, an antioxidant, a coloring agent, a humectant, a thickener, a polish, an activity enhancer, an anti-inflammatory agent, a bactericide, a corrigent, a flavoring agent, an extender, a surfactant, a dispersant, a buffer, a preservative, a sticking agent, a flavor, a coating agent, etc. In addition, the drug formulation may include appropriately a known drug ingredient. The drug ingredients may include, for example, various vitamins (preferably, vitamin B, vitamin C, vitamin E, combinations thereof (such as a combination of vitamin C and vitamin E)), amino acid or peptide and derivatives thereof, nucleic acid and derivatives thereof, saccharides and derivatives thereof, and other ingredients such as antioxidants including carotenoid, soy isoflavone, catechins, and chlorogenic acid.

A glucono-δ-lactone content in the drug formulation is usually 0.01% by mass or more, preferably 0.1% by mass or more, more preferably 0.5% by mass or more, even more preferably 1% by mass or more to the total mass of the drug formulation, and the content is 90% by mass or less, preferably 60% by mass or less. For example, the content may be from 0.01 to 90% by mass, preferably from 0.1 to 60% by mass, more preferably from 0.5 to 60% by mass, even more preferably from 1 to 60% by mass.

The food product described above may include, in addition to general food and drink, functional food products such as a food product for patients, a nutritive functional food product, a supplement food product and a food for specified health uses which have a concept of reducing or suppressing the photoaging or the skin disorder caused by ultraviolet rays incusing, for example, skin inflammation such as erythema or edemas on the skin, formation of pigmentation, exacerbation of chloasma or ephelides, reduction of stratum corneum function, reduction of skinbarrier function, reduction of skin elasticityand formation of wrinkles accompanied therewith, solar elastosis, cutis rhomboidalis nuchae and skin tumor, and which indicate the concept if necessary. The functional food products are distinguished from general food products by the indication.

The food product maybe in a form of a solid, a semi-solid, or liquid. Examples of the food product may include breads, noodles, confectioneries such as cookies, jelly, dairyproducts, frozen food products, convenience food products, starch-processed products, processed meat products, other processed food products, beverages such as carbonated drinks, fruit juice drinks, tea drinks, soft drinks, vegetable drinks, and coffee, soups, seasonings, supplements, etc., and ingredients thereof. The food product may be in a form of a tablet, a pill, a capsule, liquid, syrup, a powder, a granule, etc., as in the drug formulation for oral administration.

The food product can be prepared by appropriately combining with an arbitrary ingredient for food and drink, a solvent, a softener, oil, an emulsifier, preservative, flavor, a stabilizer, a coloring agent, an antioxidant, a moisturizing agent, a thickener, a sticking agent, a dispersant, a humectant, etc.

In addition, various vitamins (preferably, vitamin B, vitamin C, vitamin E, combinations thereof (such as a combination of vitamin C and vitamin E), amino acid or peptide and derivatives thereof, nucleic acid and derivatives thereof, saccharides and derivatives thereof, and other ingredients such as antioxidants including carotenoid, soy isoflavone, catechins, and chlorogenic acid may be appropriately admixed.

A glucono-δ-lactone content in the food and drink product varies depending on the form of use, and is usually 0.01% by mass or more, preferably 0.1% by mass or more, more preferably 0.2% by mass or more, even more preferably 0.4% by mass or more, and the content is 50% by mass or less, preferably 20% by mass or less, more preferably 10% by mass or less. For example, the content is from 0.01 to 50% by mass, preferably from 0.1 to 10% by mass, more preferably from 0.2 to 10% by mass, even more preferably from 0.4 to 10% by mass.

When the oral ultraviolet resistance enhancer of the present invention is used as a pharmaceutical product or is added to a pharmaceutical product or a food product, the dose or intake thereof may vary depending on the condition of a human, the body weight, the gender, the age and other factors, and the dose per day per adult in an oral administration is usually 0.01 g or more of glucono-δ-lactone, preferably 0.05 g or more, more preferably 1 g or more, even more preferably 2 g or more, and the dose is 10 g or less, preferably 5 g or less. The does per day per adult is, for example, from 0.01 to 10 g, preferably from 0.05 to 10 g, more preferably from 1 g to 10 g, even more preferably from 2 g to 5 g.

The drug formulation may be administered according to an arbitrary dosage regimen, and it is preferable to continuously administer the drug formulation over several weeks to several months, dividing it into once or several times per day. For example, it is preferable to continuously administer or ingest the drug formulation over a week or more, dividing it into once to ten times per day. It is more preferable to continuously administer or ingest the drug formulation over two weeks or more, dividing it into one to five times per day.

A subject to be administered or ingest is not particularly limited so long as the subject is an animal which needs or desires the administration or intake, and may include a human who needs or desires the reduction or suppression of photoaging or skin disorders induced by exposure to ultraviolet rays including, for example, skin inflammation such as erythema or edemas on the skin, formation of pigmentation, exacerbation of chloasma or ephelides, reduction of stratum corneum function, reduction of skin barrier function, reduction of skin elasticity and formation of wrinkles accompanied therewith, solar elastosis, cutis rhomboidalis nuchae, and skin tumor.

As for the embodiments describedabove, the following aspects are further disclosed in the present invention.
<1> An oral ultraviolet resistance enhancer comprising glucono-6-lactone as an active ingredient.
<2> Use of glucono-δ-lactone for producing an oral ultraviolet resistance enhancer.
<3> Glucono-δ-lactone for use in enhancement of an oral ultraviolet resistance.
<4> A method of enhancing an ultraviolet resistance comprising: orally administering or ingesting an effective amount of glucono-6-lactone.
<5> In <1> to <4> described above, the ultraviolet resistance enhancement is to reduce or suppress a skin disorder induced by exposure to ultraviolet rays.
<6> In <5> described above, the skin disorder induced by exposure to ultraviolet rays is skin inflammation or skin photoaging.
<7> In <5> described above, the skin disorder induced by exposure to ultraviolet rays is at least one disorder selected from the group consisting of skin inflammation such as erythema or edemas on the skin, pigmentation, exacerbation of chloasma or ephelides, reduction of stratum corneum function, reduction of skin barrier function, reduction of skin elasticity, solar elastosis, cutis rhomboidalis nuchae, and skin tumor.
<8> In <3> described above, the use is a non-therapeutic use.
<9> The non-therapeutic use according to <8> described above, which is use as a food product for patients, a nutritive functional food product, a supplement food product, or a food for specified health uses.
<10> In <4> described above, the method is a non-therapeutic method.
<11> The non-therapeutic method according to <10> described above, wherein the oral administration or ingestion is an oral administration or ingestion of a food product for patients, a nutritive functional food product, a supplement food product, or a food for specified health uses.
<12> In <4> described above, a subject to be administered or ingest is an animal, preferably a human, who needs or desires reduction or suppression of a skin disorder induced by exposure to ultraviolet rays, for example, skin inflammation such as erythema or edemas on the skin, pigmentation, exacerbation of chloasma or ephelides, reduction of stratum corneum function, reduction of skinbarrier function, reduction of skin elasticity, solar elastosis, cutis rhomboidalis nuchae, or skin tumor.
<13> In <4> described above, a subject to be administered or ingest is a human who does not desire application of a sunscreen external preparation, a human whose skin disagrees with a sunscreen external preparation, or a human who has a high sensitivity to ultraviolet rays.
<14> The oral ultraviolet resistance enhancer according to <1> or <2>, which is used in a dose or intake of glucono-δ-lactone per day per adult of 0.01 g or more, preferably 0.05 g or more, more preferably 1 g or more, even more preferably 2 g or more, and 10 g or less, preferably 5 g or less.
<15> The use according to any one of <3> and <5> to <9>, or the method according to any one of <4> to <7> and <10> to <13>, wherein a dose or intake of glucono-6-lactone per day per adult is 0.01 g or more, preferably 0.05 g or more, more preferably 1 g or more, even more preferably 2 g or more, and 10 g or less, preferably 5 g or less.
<16> The oral ultraviolet resistance enhancer according to any one of <1>, <2> and <5> to <7>, wherein a glucono-δ-lactone content is 0.01% by mass or more, preferably 0.1% by mass or more, more preferably 0.5% by mass or more, even more preferably 1% by mass or more, and 90% by mass or less, preferably 60% by mass or less to the total mass of the drug formulation.
<17> In the use or method according to <9> or <11>, a glucono-δ-lactone content is 0.01% by mass or more, preferably 0.1% by mass or more, more preferably 0.2% by mass or more, even more preferably 0.4% by mass or more, and 50% by mass or less, preferably 20% by mass or less, more preferably 10% by mass or less in the food product.
<18> The use or method according to <15> or <17>, wherein the administration or ingestion is performed in a continuous administration or ingestion over two weeks or more, as being divided into once to five times per day.
<19> Use of glucono-δ-lactone in a supplement food product for controlling an ultraviolet resistance of the skin.
<20> Use of glucono-δ-lactone in production of a supplement food product for controlling an ultraviolet resistance of the skin.

### EXAMPLES

### Example 1

### Effect of Suppressing Erythema and Increase of Epidermal Thickness Induced by Ultraviolet Rays

### 1) Method

HR-1 hairless mice (female, 8 weeks of age) (Japan SLC, Inc.) were bred in conditions of a temperature of 23±1° C., a humidity of 50±1%, and a lighting time of 7:00 to 19:00, and the mice freely ingested feed and water during the test period. After one-week preliminary breeding, the mice were divided into a control group, a 0.5% glucono-δ-lactone mixed feed group (0.5% GDL group), and a 1.0% glucono-δ-lactone mixed feed group (1% GDL group), and feed having a composition shown in Table 1 was given to each group for 2 weeks. After the two-week ingestion term, two sites (an irradiation site and a non-irradiation site) of 1.5 cm×1.0 cm were set in contiguity with each other on the back of the mouse under pentobarbital anesthesia, and irradiation was applied to the irradiation site in a UVB exposure dose of 1 mW/cm² for 40 seconds (40 mJ/cm²). A degree of erythema was evaluated after 2 days from the irradiation by a skin color (a*value) measurement using a spectrophotometer SE-6000 (Nippon Denshoku Industries Co., Ltd.). The a*value is an index showing redness of the skin color, and it can be said that the skin turns more red, namely, the more erythema are generated, as the a* value is increased.

After the measurement, blood was drawn from a heart under deep anesthesia, and the skin was taken from the UVB non-irradiation site and the irradiation site. After that, HE stain specimens were prepared, and the specimens were observed with a microscope. As for an epidermal thickness, an average value of epidermal thicknesses at 15 arbitrary points per tissue was defined as the epidermal thickness of the tissue. The obtained values are expressed as Ave.±S.D., n=8, and as for a statistical significance test between many groups, a multiple comparison test according to Dunnett was performed (*p<0.05, **p<0.01 (vs GDL (−) UVB (+)).

TABLE 1

| | | 0.5% | 1% |
|---|---|---|---|
| | Control | GDL | GDL |
| Corn oil (Oriental Yeast Co., Ltd.) | 50 | 50 | 50 |
| Milk casein (Oriental Yeast Co., Ltd.) | 200 | 200 | 200 |
| α-Potato starch (Oriental Yeast Co., Ltd.) | 665 | 660 | 655 |
| Cellulose powder (Oriental Yeast Co., Ltd.) | 40 | 40 | 40 |
| Mineral mixture AIN-76 (Oriental Yeast Co., | 35 | 35 | 35 |
| Ltd.) | | | |
| Vitamin mixture AIN-76 choline bitartrate | 10 | 10 | 10 |
| (Oriental Yeast Co., Ltd.) | | | |
| Glucono-δ-lactone (Sigma) | 0 | 5 | 10 |
| total (g) | 1000 | 1000 | 1000 |

### 2) Results

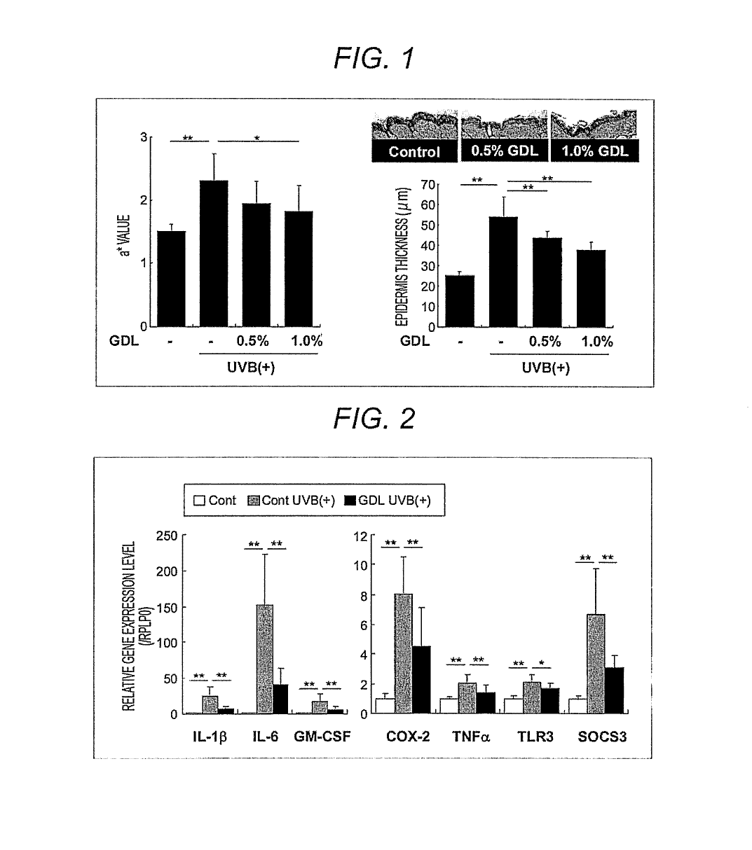

The results are shown in FIG. 1. In the GDL groups, the increase of a*values due to the UVB irradiation, was concentration-dependently suppressed; and in the irradiation sites in the 1% GDL group, the significant suppression of a* value was observed compared to the irradiation sites in the control group. In addition, in the irradiation sites in the GDL groups, the increase of epidermal thickness was significantly suppressed compared to the irradiation sites in the control group. It was revealed from the above that GDL has an effect of enhancing the ultraviolet resistance.

### Example 2

### Effect of Suppressing Expression of Ultraviolet Ray-Induced, Inflammation-Related Molecule, Melanin Synthesis-Related Molecule, Proliferation-Related Molecule, and Dermal Denaturation Factor

### 1) Method

HR-1 hairless mice (female, 8 weeks of age) (Japan SLC, Inc.) were bred in conditions of a temperature of 23±1° C., a humidity of 50±1%, and a lighting time of 7:00 to 19:00, and the mice freely ingested feed and water during the test period. After one-week preliminary breeding, the mice were divided into a control group and a 1.0% GDL group, and feed having a composition shown in Table 1 was given for 2 weeks. After the two-week ingestion term, two sites (an irradiation site and a non-irradiation site) of 1.5 cm×1.0 cm were set in contiguity with each other on the back of the mouse under pentobarbital anesthesia, and irradiation was applied to the irradiation site in a UVB exposure dose of 1 mW/cm² for 40 seconds (40 mJ/cm²). After 24 hours from the irradiation, blood was drawn from a heart, and the skin was taken from the UVB non-irradiation site and the irradiation site, from which RNA was extracted. A gene expression level of the molecule, shown below, was quantified from the extracted RNA according to qRT-PCR. As a TaqMan Gene Expression Assay (Applied Biosystems) probe, which specifically detects a target gene, were used Il-1b (Mm01336189_m1; IL-1b, Il-6 (Mm00446190_m1; IL-6), Gm-csf (Mm01290062_m1; GM-CSF), Tnf (Mm00443258_m1; TNFa), Ptgs2 (Mm01307329_m1; COX-2), Tlr3 (Mm01207404_m1; TLR3), Socs3 (Mm00545913_s1; SOCS3), Vcam1 (Mm01320970_m1; VCAM-1), Icam1 (Mm00516023_m1; ICAM-1), Sele (Mm00441278_m1; E-selectin), Edn1 (Mm00438656_m1; EDN1), Kit1 (Mm00442972_m1; SCF), C-kit (Mm00445212_m1; c-Kit), Lif (Mm00434761_m1; LIF), Fgf2 (Mm00433287_m1; FGF2), Hgf (Mm01135185_m1; HGF), Pcna (Mm00448100_g1; PCNA), Ccnd1 (Mm00432359_m1; CyclinD1), Mmp13 (Mm00439491_m1; MMP13), Mmp2 (Mm00439506_m1; MMP2), Mmp9 (Mm00442991_m1; MMP9), and Mmp14(Mm00485054_m1; MMP14). A target gene expression level was corrected by an expression level of an internal standard gene Rplp0 (Mm01974474_gH; RPLP0). An analysis was performed using 7500 Fast Real-Time PCR System (Applied Biosystems). The obtained values are expressed as Ave.±S.D., n=10, and as for a statistical significance test between many groups, a multiple comparison test according to Dunnett was performed (*p<0.05, **p<0.01 (vs Cont UVB+).
IL-1β: Interleukin-1β
IL-6: Interleukin-6
GM-CSF: Granulocyte macrophage colony-stimulating factor
TNFα: Tumor necrosis factor α
COX-2: Cyclooxygenase-2
TLR3: Toll-like receptor 3
SOCS3: Suppressor of cytokine signaling 3
VCAM-1: Vascular cell adhesion molecule-1
ICAM-1: Intercellular adhesion molecule-1
E-selectin
EDN1: Endothelin 1
SCF: Stem cell factor (kit-ligand)
c-Kit
LIF: Leukemia inhibitory factor
FGF2: Fibroblast growth factor 2 (basic)
HGF: Hepatocyte growth factor
PCNA: Proliferating cell nuclear antigen
CyclinD1
MMP13: Matrix metallopeptidase 13 (Collagenase 3)
MMP2: Matrix metallopeptidase 2 (Gelatinase A)
MMP9: Matrix metallopeptidase 9 (Gelatinase B)
MMP14: Matrix metallopeptidase 14 (membrane-inserted)

### 2) Results

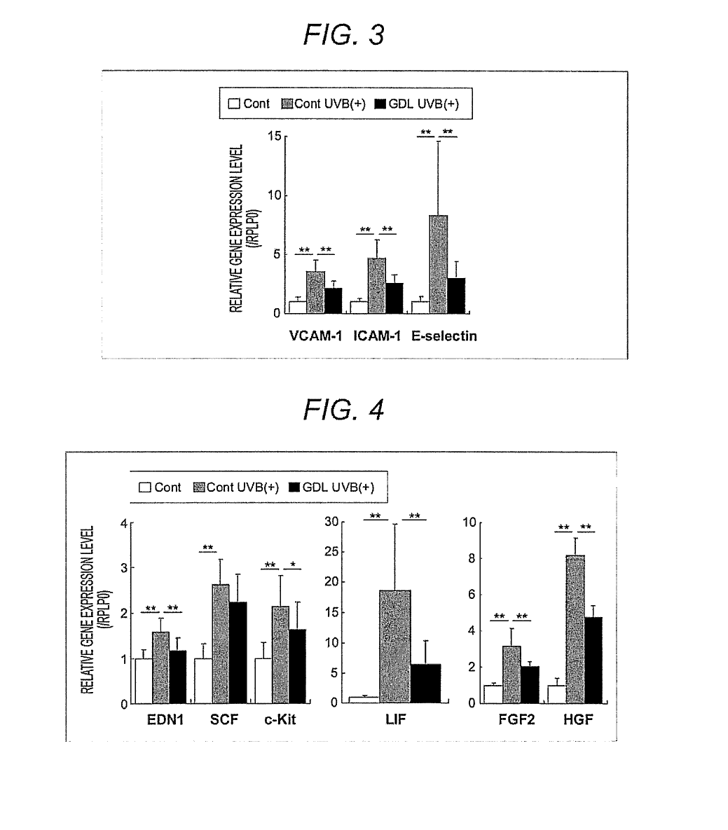

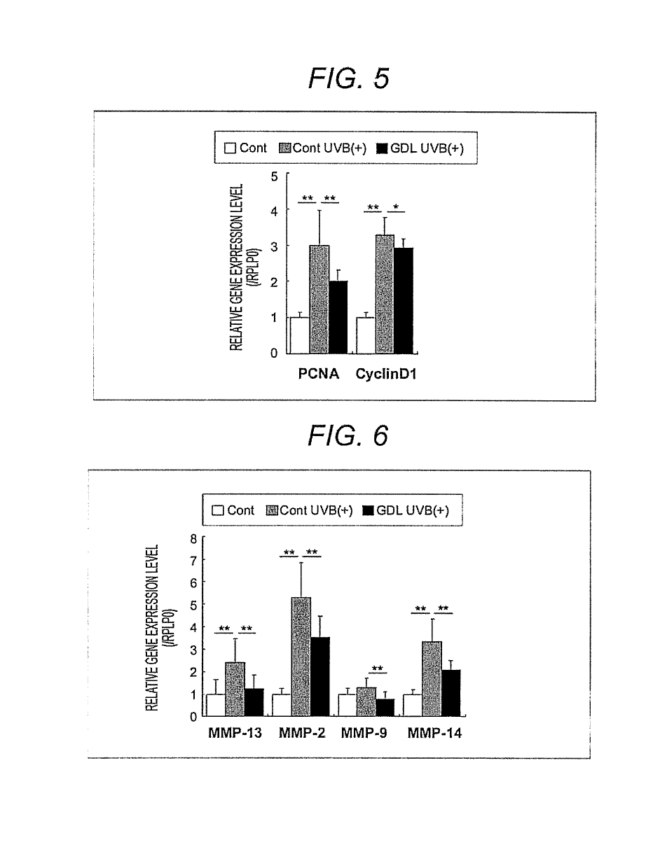

The results are shown in FIGS. 2 to 6. The UVB irradiation increased the gene expressions of IL-1β, IL-6, GM-CSF, TNFα, COX-2, TLR3, and SOCS3. It was revealed that the expressions of the inflammation-related genes in the irradiation sites in the GDL group were significantly suppressed compared to the irradiation sites in the control group. In addition, it was observed that the expressions of the adhesion molecules VCAM-1, ICAM-1, and E-selectin, induced upon the inflammation, were also suppressed in the GDL group. As the expression analysis of the melanin synthesis-related molecules was similarly performed, the expressions of EDN1, c-Kit, LIF, FGF2, and HGF were increased by UVB, and it was revealed that the expressions were significantly reduced in the GDL group. Further, the expressions of PCNA and CyclinD1, which are markers of cell proliferation, were also suppressed. It was also observed that the expressions of collagenase (MMP13), gelatinase (MMP2, and MMP9), membrane-associated MMP (MMP14), which are suggested to be related to dermal denaturation, were suppressed in the GDL ingestion group.

### Example 3

### Effects of Suppressing Pigmentation, Reduction of Skin Stratum Corneum Function, and Reduction of Skin Elasticity, Caused by Ultraviolet Rays

### 1) Method

Grouping of HRM-2 hairless mice (male, 6 weeks of age) (Japan SLC, Inc.) was performed so as to equalize a body weight, a lightness (L*value), a degree of red color (a*value), a transepidermal water loss (a TEWL value), and a stratum corneum moisture (Capacitance) to obtain three groups of a control non-irradiation group (Cont), a control UVB irradiation group (Cont (UVB+)), and a 2.0% GDL UVB irradiation group (GDL (UVB+)). After a test feed was previously ingested for 3 weeks, UVB irradiation was applied to the UVB irradiation groups once per day over 20 weeks. As an ultraviolet resistance was obtained by continuous irradiation, a UVB irradiation intensity was increased from 40 mJ/cm² to 130 mJ/cm² in stages (irradiation in 0 to the first week: 40 mJ/cm², irradiation in the second to the forth weeks: 54 mJ/cm², irradiation in the fifth to the seventh weeks: 72 mJ/cm², irradiation in the eighth to the twelfth weeks: 108 mJ/cm², irradiation in the thirteenth to the fourteenth weeks: 120 mJ/cm², irradiation in the fifteenth to twentieth weeks: 130 mJ/cm²). During the UVB irradiation term, the test feed was ingested. At the time of the grouping, and the irradiation 0th, 4th, 8th, 12th, 16th, and 20th weeks, the measurements of L*values using a spectrophotometer, TEWL values using a Tewameter, and stratum corneum moisture contents (the Capacitance value) using a Corneometer were performed. At the irradiation 20th week, skin physical parameters were measured using a Cutometer. The obtained values are expressed as Ave.±S.D., n=10-11, and the multiple comparison test was performed according to Tukey-Kramer.

### 2) Results

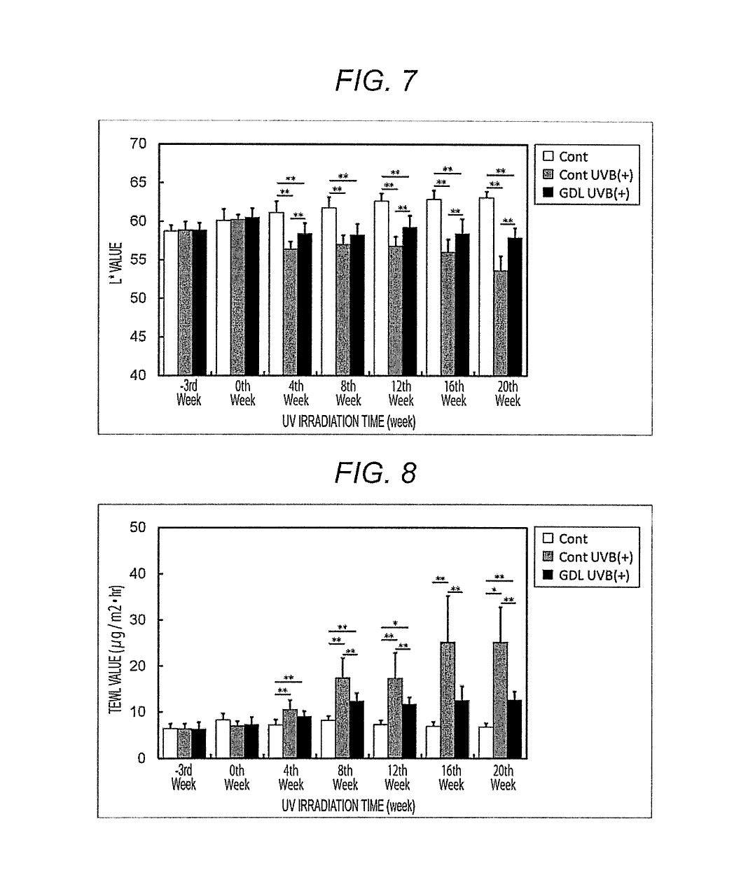

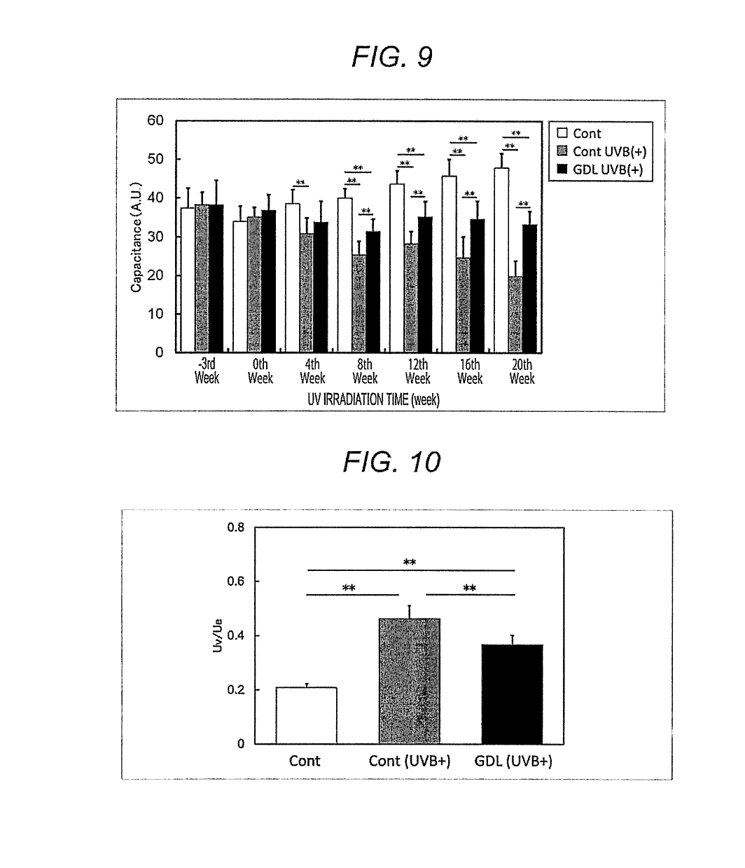

The results are shown in FIGS. 7 to 10. After the UV irradiation 8th week or 12th week, the decrease of L*value, the increase of TEWL value, and the decrease of stratum corneum moisture content (the Capacitance value) caused by the UVB irradiation were suppressed by the GDL ingestion. Further, the reduction of the skin viscoelasticity (Uv/Ue) was suppressed. It was revealed from the above that GDL suppressed various skin disorders, caused by a long-term repeated ultraviolet ray irradiation.

### Example 4

### Effect of Suppressing Ultraviolet Ray-Induced Erythema and Pigmentation in Human 1

### 1) Method

Ten healthy males (twenties to forties age) were subjected to a crossover test in which the same test participant continuously ingested a capsule including glucono-δ-lactone (an intake per day was 2000 mg of glucono-δ-lactone) or a placebo capsule over 4 weeks in different periods. A 4-week ingestion interval was provided between the first half and the last half of the crossover test. Glucono-δ-lactone, which is a test product, is a food additive (Fuso Chemical Co., Ltd.), and the intake was divided into twice a day for ingestion. After a 3-week ingestion term, irradiation was applied to an upper arm inside part with a UVB intensity of 1 mW/cm² for 7 different time periods, each at different sites (an irradiation area of 0.6 cm×1.0 cm per site). After 24 hours from the UVB irradiation, an MED value (the minimum erythema dose) was visually evaluated.

After the 3-week ingestion term, irradiationwith a radiation dose corresponding to 2 MED was applied to an area of 1.5 cm×1.5 cm on the upper arm inside part with a UVB intensity of 1 mW/cm². After 24 hours from the UVB irradiation, a ΔAa*value (erythema intensity: a difference from that in the non-irradiation site) was measured using a spectrophotometer. Further, after one week from the UVB irradiation, a ΔL*value (degree of pigmentation: a difference from that in the non-irradiation site) in each irradiation site wasmeasuredusing the spectrophotometer. The obtained values are expressed as Ave.±S.D., n=10, and as for a statistical significance test between the two groups, a paired t-test was performed (*p<0.05, **p<0.01).

### 2) Results

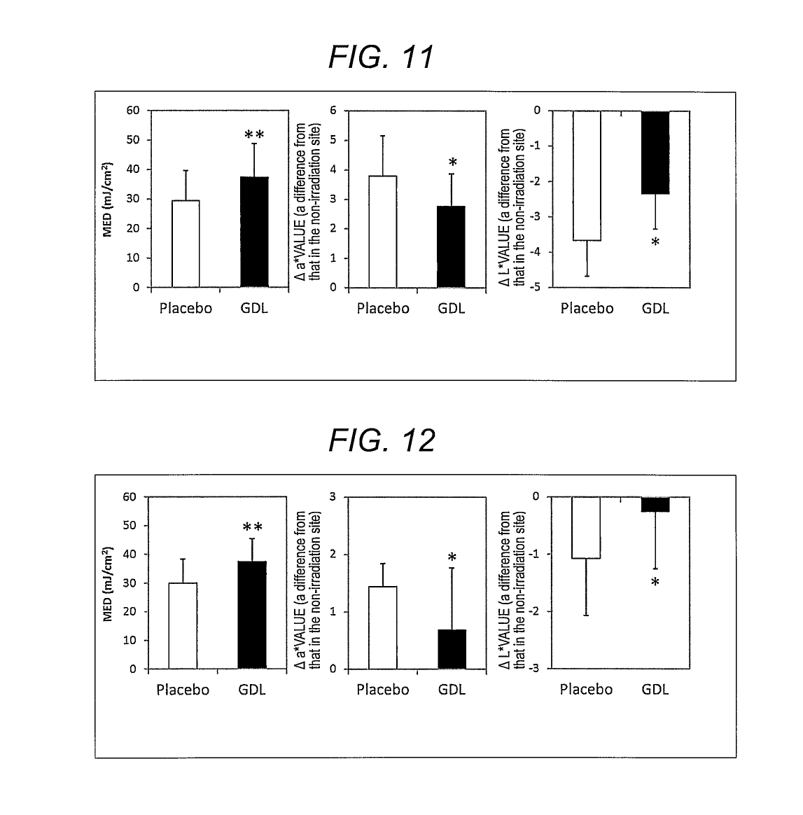

The results are shown in FIG. 11. In the GDL group, the MED values were significantly high compared to the placebo group, and it was revealed that the ultraviolet resistance was enhanced. In fact, the Δa*values after 24 hours from the UVB irradiation in the GDL group were significantly decreased compared to the placebo group, and formation of the ultraviolet ray-induced erythema was suppressed. In addition, the AL* values after one week from the UVB irradiation in the GDL group were also significantly decreased compared to the placebo group, and it was revealed that the pigmentation was suppressed.

### Example 5

### Effect of suppressing Ultraviolet Ray-Induced Erythema and Pigmentation in Human 2

### 1) Method

Ten healthy males (twenties to forties age) were subjected to a crossover test in which the same test participant continuously ingested a capsule including glucono-δ-lactone and vitamins (an intake per day was 2000 mg of glucono-δ-lactone, 200 mg of d-α-tocopherol, and 666 mg of L-ascorbic acid) or a placebo capsule over 4 weeks in different periods. A 4-week ingestion interval was provided between the first half and the last half of the crossover test. Glucono-δ-lactone, which is a test product, is a food additive (Fuso Chemical Co., Ltd.), and an intake was divided into twice a day for ingestion. After a 3-week ingestion term, irradiation was applied to an upper arm inside part in a UVB irradiation dose of 1 mW/cm² for 7 different time periods, each at different sites (an irradiation area of 0.6 cm×1.0 cm per site).

After 24 hours from the UVB irradiation, an MED value (minimum erythema dose) was visually evaluated, and a Δa* value (erythema intensity: a difference from that in the non-irradiation site) was measured in each irradiation site by taking photographs and using a spectrophotometer. Further, after one week from the UVB irradiation, a ΔL* value (degree of pigmentation: a difference from that in the non-irradiation site) in each irradiation site was measured by taking photographs and using the spectrophotometer. As the Δa* values and the ΔL*values, results in the UV dose decided as 1 MED by each subject upon the placebo ingestion are shown. The obtained values are expressed as Ave.±S.D., n=10, and as for a statistical significance test between the two groups, a paired t-test was performed (*p<0.05, **p<0.01).

### 2) Results

The results are shown in FIG. 12. In the GDL group, the MED values were significantly high compared to the placebo group, and it was revealed that the ultraviolet resistance was enhanced. In fact, the Δa*values after 24 hours from the UVB irradiation in the GDL group were significantly decreased compared to the placebo group, and formation of the ultraviolet ray-induced erythema was suppressed. In addition, the ΔL* values after one week from the UVB irradiation in the GDL group were also significantly decreased compared to those in the placebo group, and it was revealed that the pigmentation was suppressed.

What is claimed is:

1.-12. (canceled)

13. A method of enhancing an ultraviolet resistance comprising:
orally administering or ingesting an effective amount of glucono-δ-lactone.

14. The method according to claim 13, wherein the enhancing is reduction or suppression of a skin disorder induced by exposure to ultraviolet rays.

15. The method according to claim 14, wherein the skin disorder induced by exposure to ultraviolet rays is skin inflammation or skin photoaging.

16. The method according to claim 14, wherein the skin disorder induced by exposure to ultraviolet rays is at least one disorder selected from the group consisting of skin inflammation pigmentation, exacerbation of chloasma or ephelides, reduction of stratum corneum function, reduction of skin barrier function, reduction of skin elasticity, solar elastosis, cutis rhomboidalis nuchae and skin tumor.

17. The method according to claim 14, wherein the glucono-δ-lactone is administered to or ingested by an animal or a human subject who needs or desires reduction or suppression of the skin disorder induced by exposure to ultraviolet rays and the skin disorder induced by exposure to ultraviolet rays is at least one disorder selected from the group consisting of skin inflammation, pigmentation, exacerbation of chloasma or ephelides, reduction of stratum corneum function, reduction of skin barrier function, reduction of skin elasticity, solar elastosis, cutis rhomboidalis nuchae and skin tumor.

18. The method according to claim 13, wherein the glucono-δ-lactone is administered to or ingested by a human subject who does not desire application of a sunscreen external preparation, a human subject whose skin disagrees with a sunscreen external preparation, or a human subject who has a high sensitivity to ultraviolet rays.

19. The method according to claim 1, wherein the glucono-δ-lactone is administered or ingested at a dose or intake of 0.01 g or more and 10 g or less glucono-δ-lactone per day per adult.

20. The method according to claim 1, wherein the glucono-δ-lactone is administered or ingested at a dose or intake of 0.05 g or more and 10 g or less glucono-δ-lactone per day per adult.

21. The method according to claim 1, wherein the administering or ingesting is performed in a continuous administration or ingestion over two weeks or more, as being divided into once to five times per day.
